(19) United States
(12) Patent Application Publication
(10) Pub. No.: US 2013/0184559 A1
(43) Pub. Date: Jul. 18, 2013

(54) Magnetic Resonance Imaging Compatible Positron Emission Tomography Detector

(71) Applicants: James L. Corbeil, Knoxville, TN (US); Nan Zhang, Knoxville, TN (US); Matthias J. Schmand, Lenoir City, TN (US)

(72) Inventors: James L. Corbeil, Knoxville, TN (US); Nan Zhang, Knoxville, TN (US); Matthias J. Schmand, Lenoir City, TN (US)

(73) Assignee: Siemens Medical Solutions USA, Inc., Malvern, PA (US)

(21) Appl. No.: 13/740,364

(22) Filed: Jan. 14, 2013

Related U.S. Application Data
(60) Provisional application No. 61/585,816, filed on Jan. 12, 2012.

(51) Int. Cl. A61B 5/00

(52) U.S. Cl. CPC A61B 5/0035
USPC 600/411

(57) ABSTRACT

A compact magnetic resonance imaging compatible positron emission tomography detector. The detector has integrated mechanical and electrical subcomponents. The detector uses a cooling channel which does not interfere with magnetic resonance imaging. The layout and selection of electrical subcomponents of the detector, along with a magnetic resonance compatible cooling strategy, enables the detector to function in a magnetic resonance imaging environment.

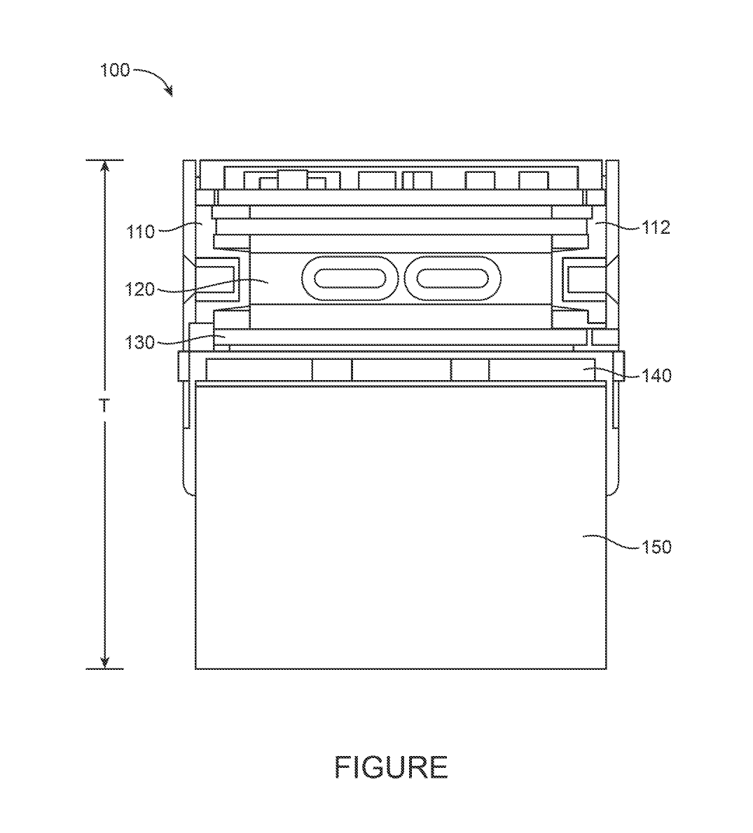

### CROSS-REFERENCE TO RELATED APPLICATION

This application claims priority to U.S. Provisional Application No. 61/585,816, entitled, A Compact MRI Compatible PET Detector, which was filed on Jan. 12, 2012 and which is fully incorporated by reference herein.

### FIELD OF TECHNOLOGY

The present disclosure relates generally to Positron Emission Tomography (PET) detectors, and more particularly to a compact Magnetic Resonance Imaging (MRI) compatible PET detector.

### BACKGROUND

Current PET detector designs, for both PET and combined Positron Emission Tomography-Computed Tomography (PET/CT) applications, are generally not limited by environmental and geometric factors, but instead evolve around the inner workings of the detectors. For example, the form factors of PET/CT detectors are generally designed around the heights of a detector's internal components, including the Lutetium Oxyorthosilicate (LSO) array, Photomultiplier Tube (PMT) array, and bleeder Printed Circuit Board (PCB). The detector, as a fundamental building block of a larger system, generally dictated the subsequent spacing for its surrounding structures. Other than shielding for changes in the Earth's magnetic field and ensuring that the detector was impermeable with regard to light, few external factors were allowed to affect the designs of detectors. However, with the advent of combined Magnetic Resonance Imaging-PET (MR/PET) imaging detectors, designs must be highly integrated and “smart” in order to obtain PET imaging data within the unique environment of a MRI apparatus.

### BRIEF DESCRIPTION OF THE DRAWINGS

In order to describe a manner in which features of the disclosure can be obtained, reference is made to specific embodiments that are illustrated in the appended drawing. Based on an understanding that the drawing depicts only an example embodiment of the disclosure and is not intended to be limiting of scope, the principles herein are described and explained with additional specificity and detail through the use of the accompanying drawing in which:

The FIGURE illustrates an example embodiment of a magnetic resonance imaging compatible positron emission tomography detector.

### DETAILED DESCRIPTION

Various aspects of the disclosure are discussed in detail below. While specific implementations are discussed, it should be understood that this is done for illustration purposes only.

The FIGURE illustrates an example embodiment of a magnetic resonance imaging compatible positron emission tomography (MRI/PET) detector 100. The components of the detector 100, discussed in greater detail below, are highly integrated and “smart” in order to interact and survive in the unique magnetic resonance imaging (MRI) environment.

The detector 100 incorporates highly optimized radio frequency (RF) shielding, an internalized cooling strategy, and highly specialized printed circuit boards, all of which are packaged in an envelope three times shorter in height of thickness than most detectors. In addition to being shorter than other detectors, the example detector 100 illustrated meets the operational requirements of other position emission tomography (PET) detectors.

The detector 100 includes several subcomponents which are collectively integrated. These subcomponents can include a PCB array 130, a thermally optimized avalanche photodiode (APD) seat 140, MR compatible radiation shielding, a highly optimized cooling channel 120, thin RF screening, and a lutetium oxyorthosilicate array 150. The PCB array 130 can be flexible or rigid or flexible and rigid. In at least one embodiment, each subcomponent plays at least a dual role in order to achieve hybridization and integration.

A Rigid-Flex PCB array 130 can obviate the need for connectors and can minimize overall stack height of the detector, enabling the detector 100 to fit into a height envelop of less than 45 mm, while withstanding the vibration loads to which the PCB array 130 may be subjected during normal use. The PCB array 130 is designed and configured to be reduce sensitivity to gradient and radio frequency pulses, and is optimized for heat transfer. The flexible regions 110, 112 of the detector 100, along with the absence of connectors, enable the PCB array 130 to be foldable around a cooling channel 120, thereby maximizing the thermal contact area.

A thermally optimized seat 140 functions to locate the APD and lutetium oxyorthosilicate (LSO) arrays 150. The APD seat 140 can be molded with a thermally conductivity polymer. The APD seat can include embedded ceramics to aid in heat conduction. The seat 140 structure is exceptionally thin, but even so, it is able to anchor the front, back, and sides of the detector 100, as well as serving to position a tungsten polymer radiation shielding. The seat 140 can also serve as a light reflector for the APD and LSO arrays 150.

At least one embodiment of the detector 100 includes radiation shielding. The radiation shielding can be comprised of a tungsten polymer which can shield against up to 511 kiloelectron volts (keV). The tungsten polymer shield does not distort magnetic fields. The tungsten polymer can be injection moldable, thereby making it possible to mold the shielding and further hybridize the detector 100.

At least one embodiment of the detector 100 includes an optimized cooling channel 120. The cooling channel 120 can serve as the main heat transfer mechanism for transferring heat away from and out of the detector 100. The cooling channel 120 can provide strength and rigidity, thereby enabling the channel to serve as the backbone for the detector 100. The cooling channel 130 is optimized to maximize heat transfer, while minimizing the effects of eddy currents and Lorenz Forces. This optimization can be accomplished by appropriate selection and shaping of copper, ceramics and polymers into a design which is only about four millimeters thick. With appropriate selection and shaping of the cooling channel 130 materials, the detector can be capable of withstanding nineteen times normal barometric pressure.

At least one embodiment includes thin RF shielding comprising a RF screen. The thin RF shielding provides adequate screening of RF and light impermeability and light tightness for the detector 100. The RF screen is unaffected by vibrations and Lorenz forces. The RF screen prevents spiking artifacts and damage in the MRI image produced by the detector 100. The RF screen utilizes a subcomponent structure and takes advantage of an adhesive backing of the copper foil therein.

At least one embodiment of the MRPET of this disclosure includes at least one flex region 110, 112, a cooling channel 120 coupled to the at least one flex region 110, 112, at least one PCB array 130 coupled to the cooling channel 120, at least one APD array coupled to the at least one PCB array, and at least one LSO array 150 coupled to the avalanche photodiode array. As discussed above, these subcomponents can be integrated such that a thickness T of the detector 100 is smaller than 45 millimeters. In at least one embodiment, the detector 100 subcomponents can be integrated such that a thickness T of the detector 100 is smaller than 42 millimeters.

In at least one embodiment of the detector 100, an APD array is housed within a thermally optimized seat 140 in order to maximize heat transfer away from the detector 100. As discussed above, the thermally optimized seat can comprise a thermally conductive polymer.

In at least one embodiment of the detector 100, the cooling channel 120 comprises copper and at least one ceramic material, such that the cooling channel 120 has a thickness of smaller than five millimeters. In at least one embodiment, the cooling channel 120 has a thickness of four millimeters. As discussed above, the cooling channel 120 is configured so as to not interfere with a magnetic resonance signal.

In at least one embodiment of the disclosure, the detector 100 can be a cassette detector.

By intensively integrating both the mechanical and the electrical subcomponents discussed above, as well as optimizing the layout and selection of electrical components, and by having a MR compatible cooling strategy, the detector 100 is able to both operate and survive an MRI environment.

The various embodiments described above are provided by way of illustration only and should not be construed to limit the scope of the disclosure. It will be understood that modifications and changes may be made using the principles described herein without departing from the scope of the disclosure or the following claims.

What is claimed is:

1. A magnetic resonance imaging compatible positron emission tomography detector comprising:
at least one flex region;
a cooling channel coupled to the at least one flex region;
at least one printed circuit board array coupled to the cooling channel;
at least one avalanche photodiode array coupled to the at least one printed circuit board array; and
at least one lutetium oxyorthosilicate array coupled to the avalanche photodiode array.

2. The detector as recited in claim 1, wherein a thickness of the detector is smaller than 45 millimeters.

3. The detector as recited in claim 1, wherein a thickness of the detector is smaller than 42 millimeters.

4. The detector as recited in claim 1, wherein the at least one avalanche photodiode array is housed within a thermally optimized seat.

5. The detector as recited in claim 4, wherein the thermally optimized seat comprises a thermally conductive polymer.

6. The detector as recited in claim 1, further comprising a radiation shield.

7. The detector as recited in claim 6, wherein the radiation shield comprises a tungsten polymer.

8. The detector as recited in claim 1, wherein the cooling channel comprises copper and at least one ceramic material.

9. The detector as recited in claim 8, wherein a thickness of the cooling channel is smaller than five millimeters.

10. The detector as recited in claim 1, wherein the cooling channel is configured so as to not interfere with a magnetic resonance signal.

11. The detector as recited in claim 1, wherein the detector is a cassette detector.
